(19) United States
(12) Patent Application Publication
(10) Pub. No.: US 2010/0286056 A1
(43) Pub. Date: Nov. 11, 2010

(54) COMPOSITIONS FOR REDUCING OXIDATIVE STRESS AND USES THEREOF

(76) Inventor: Markus Böhm, Munster (DE)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 12/742,812

(22) PCT Filed: Nov. 19, 2008
(86) PCT No.: PCT/EP2008/065841

§ 371 Date: May 13, 2010

(30) Foreign Application Priority Data
Nov. 19, 2007 (EP) 07022401.9

(51) Int. Cl. A61K 8/64; A61K 38/06; A61P 17/14; A61P 17/18; A61Q 5/00; A61Q 19/08; A61P 39/06

(52) U.S. Cl. 514/18.8; 514/18.6; 514/20.7

(57) ABSTRACT

The present invention relates to the use of one or more tripeptides selected from the group consisting of ^{N}Lys-Pro-Val^{C}, ^{N}Lys-Pro-Thr^{C} and ^{N}pGLu-His-Pro^{C} for the reduction of oxidative stress. The above tripeptides are particularly useful for the treatment of a disease or damage caused by oxidative stress; such as vitiligo, scleroderma, necrosis, or erythema; furthermore, a disease or damage of the hair, like premature hair loss or premature formation of grey hair. Furthermore the invention relates the cosmetic use of the above tripeptides, in particular against skin aging. Further the invention relates cosmetic compositions containing at least one of said tripeptides.

The present invention relates to the use of at least one compound selected from the group consisting of ^{N}Lys-Pro-Val^{C}, ^{N}Lys-Pro-Thr^{C} and ^{N}pGlu-His-Pro^{C} for the reduction of oxidative stress, the therapeutic use of the above tripeptides for the treatment of a disease or damage caused by oxidative stress, and the cosmetic use of the above tripeptides, in particular against skin aging. Further provided by the invention are cosmetic compositions containing at least one of said tripeptides.

### BACKGROUND ART

Care products containing tripeptides as by-products to provide protective anti-inflammatory effects are known in the art. For example, DE 10 2005 022 626 A1 discloses compositions which contain taurine as an active compound and further contain peptides, such as Lys-Pro-Val as an anti-inflammatory agent. It has been shown that the tripeptide Lys-Pro-Val has anti-inflammatory properties. For instance, WO 88/00833 discloses the use of the tripeptide Lys-Pro-Val for producing a medicament for the treatment of inflammations. Several studies further suggest that the C-terminal region of αMSH (amino acids 11-13, Lys-Pro-Val) mediates the anti-inflammatory effects of αMSH (Catania and Lipton, 1993, Endocr. Rev. 14, 564-576; Bhardvaj et al., 1996, J. Immunol., 156, 2517-2521). The C-terminal tripeptide of αMSH has further been proposed as agent to prevent loss of hair (FR 2 733 421) also published as (U.S. Pat. No. 5,739,111). WO 02/064131 discloses that the tripeptide Lys-Pro-Thr and even smaller compounds, like Lys-Pro and Lys have anti-inflammatory properties.

With respect to skin aging, a distinction is made between the so-called “intrinsic” and “extrinsic” aging, a decisive factor for the latter being the exogenous effect, in particular the effect of ultraviolet (UV) radiation (“photoaging”). Mechanistically, oxidative stress plays a major role in both intrinsic and extrinsic aging of the skin since reactive oxygen species (ROS) are generated in the process of normal cellular metabolism or by physiological processes and, in addition, are produced in particular by the UVA component but also by the UVB component. If not suppressed upon formation, ROS have far-reaching effects on the integrity of all cellular bio-molecules such as DNA, protein and lipids with regard to UV-induced aging of the skin. An immediate consequence, which is of particular importance as to photoaging, is the ROS-induced induction of matrix metalloproteases which, in turn, facilitates collagen metabolism and thus increases degradation and, finally, leads to skin thinning. Thus, so far strategies for preventing photoaging consist in a reduction of UV-exposure, physical protection or the application of specific vitamins such as vitamin C or vitamin E.

It thus follows that the technical problem underlying the present invention is to comply with the need described above. Particularly, the technical problem underlying the present invention is to provide means and methods for the reduction of oxidative stress, particular to the reduction of (intracellular) ROS. The solution to this technical problem is achieved by providing the embodiments characterized in the claims.

However, as evident from the above, none of the prior art documents teach or suggest the use of tripeptides, like ^{N}Lys-Pro-Val^{C}, ^{N}Lys-Pro-Thr^{C} and ^{N}pGlu-His-Pro^{C} for the reduction of oxidative stress. Thus, there is a need for compositions comprising the above tripeptides and effective strategies for their therapeutic and cosmetic use for reducing oxidative stress; in particular for the reduction of skin aging.

### SUMMARY OF THE INVENTION

The antioxidative protection of peptides was examined with a “read-out” system of UV-aging, i.e. the intracellular amplification of oxidative stress in human dermal fibroblasts. All peptides are small molecular tripeptides that, due to their resulting molecular weight (MW<500), have promising properties with regard to transcutaneous application. Firstly, it is the tripeptide Lys-Pro-Val (KPV) exhibiting 100% sequence homology to the last 3 amino acids of the C-terminal end of α-melanocyte-stimulating hormone (alpha-MSH). Secondly, a derivative of KPV, Lys-D-Pro-Thr (KPT), was tested, which exhibits a substitution of the 3^{rd} amino acid vis-à-vis KPV and, in addition, exhibits a stereochemical modification of the 2^{nd} amino acid. Thirdly, the naturally occurring thyrotropin-releasing hormone (TRH, protirelin, thyroliberin, pGlu-His-Pro) was tested. A feature common to all three is the fact that they are natural tripeptides or tripeptides derived by modification from natural, endogenously produced hormones.

It has surprisingly been found by the inventors, that the tripeptides Lys-Pro-Val (KPV), Lys-D-Pro-Thr (KPT) and Thyrotropin Releasing Hormones (TRH, Protirelin, Thyoliberin, pGlu-His-Pro) show antioxidative protective effects in human dermal fibroblasts.

Accordingly, in a first aspect the invention relates to the use of at least one compound selected from the group consisting of ^{N}Lys-Pro-Val^{C}; ^{N}Lys-Pro-Thr^{C}; and ^{N}pGlu-His-Pro^{C} for the preparation of a composition for the reduction of oxidative stress.

For the purposes of the present invention the term “at least one compound” means either one of the compounds ^{N}Lys-Pro-Val^{C}; ^{N}Lys-Pro-Thr^{C}; and ^{N}pGlu-His-Pro^{C} alone or in combinations of two or three compounds, like
^{N}Lys-Pro-Val^{C} and ^{N}Lys-Pro-Thr^{C};
^{N}Lys-Pro-Thr^{C} and ^{N}pGlu-His-Pro^{C};
^{N}Lys-Pro-Val^{C} and ^{N}pGlu-His-Pro^{C}; and
^{N}Lys-Pro-Val^{C}, ^{N}Lys-Pro-Thr^{C} and ^{N}pGlu-His-Pro^{C}.

The term “comprising” as used in the present invention also includes the term “consisting of”.

In the context of the application the abbreviations “^{N}” and “^{C}” have the meaning “N-terminus” and “C-terminus” as usually understood in the art. In the context of the invention, it is furthermore envisaged to reverse the C- and N-terminus of the compounds of the invention.

The amino acids constituting the tripeptides of the invention, lysine, proline, valine, threonine and glutamic acid are denoted by their symbols according to the commonly used three-letter code. The term “pGlu” represents a pyroglutamyl residue and is also denoted as “Pyr”. pGlu-His Pro is also known in the art as TRH or Pyr-His-Pro. The term TRH refers to Thyreotropin Releasing Hormons also known as protirelin, thyroliberin or its chemical denomination L-Pyroglutamyl-L-histidyl-L-prolinamid.

Naturally occurring amino acids usually have the (L) configuration. However, the amino acids of the compounds used according to the present invention may have either the (L) or (D) configuration. Possible compounds of the KPV structure are (L)Lys-(L)Pro-(L)Val; (D)Lys-(L)Pro-(L)Val; (L)Lys-(D)Pro-(L)Val; (L)Lys-(L)Pro-(D)Val; (D)Lys-(D)Pro-(L)Val; (L)Lys-(D)Pro-(D)Val; (D)Lys-(L)Pro-(D)Val; and (D)Lys-(D)Pro-(D)Val. Possible compounds of the KPT structure are (L)Lys-(L)Pro-(L)Thr; (D)Lys-(L)Pro-(L)Thr; (L)Lys-(D)Pro-(L)Thr; (L)Lys-(L)Pro-(D)Thr; (D)Lys-(D)Pro-(L)Thr; (L)Lys-(D)Pro-(D)Thr; (D)Lys-(L)Pro-(D)Thr; and (D)Lys-(D)Pro-(D)Thr. Possible compounds of the TRH structure are (L)pGlu-(L)His-(L)Pro; (D)pGlu-(L)His-(L)Pro; (L)pGlu-(D)His-(L)Pro; (L)pGlu-(L)His-(D)Pro; (D)pGlu-(D)His-(L)Pro; (L)pGlu-(D)His-(D)Pro; (D)pGlu-(L)His-(D)Pro; and (D)pGlu-(D)His-(D)Pro. In a preferred embodiment of the invention the compound is selected from the group consisting of (L)Lys-(L)Pro-(L)Val; (L)Lys-(D)Pro-(L)Thr; and (L)pGlu-(L)His-(L)Pro.

The compound according to the invention may be chemically modified, preferably at the N-terminus and/or C-terminus; more preferably the compound is acetylated at the N-terminus and/or amidated or esterified at the C-terminus. Other chemical modifications of the compounds of the invention such as alkylation (e.g., methylation, propylation, butylation), arylation, etherification and esterification may be possible and are also envisaged. It is preferred that the mentioned modifications do not significantly alter the advantageous capabilities of the compounds of the invention as described herein, i.e. the chemically modified compounds of the invention have capabilities which are comparable with the capabilities of the compounds which were evaluated in the appended examples. “Comparable” is explained herein below.

It may be necessary, for reasons of resistance to degradation, to employ a protected form of the compounds of the invention. The nature of the protecting group must obviously be a biologically compatible form. Many biologically compatible protective groups are suitable, such as, for example, those provided by acylation or acetylation of the amino-terminal end or amidation of the carboxy-terminal end.

Thus, the invention also features the compounds of the invention in a protected or unprotected form. Protective groups based either on acylation or acetylation of the amino-terminal end or on amidation of the carboxy-terminal end or, alternatively, on both, are the preferred.

Further protective groups known per se are likewise possible. The modifications may also affect the amino group in the side chains of the amino acids. As stated above, it is preferred that these modifications do not significantly alter the advantageous capabilities of the compounds of the invention as described herein.

In a more preferred embodiment of the invention the above tripeptides are amidated at the C-terminus; for instance compound pGlu-His-Pro is amidated at the C-terminus.

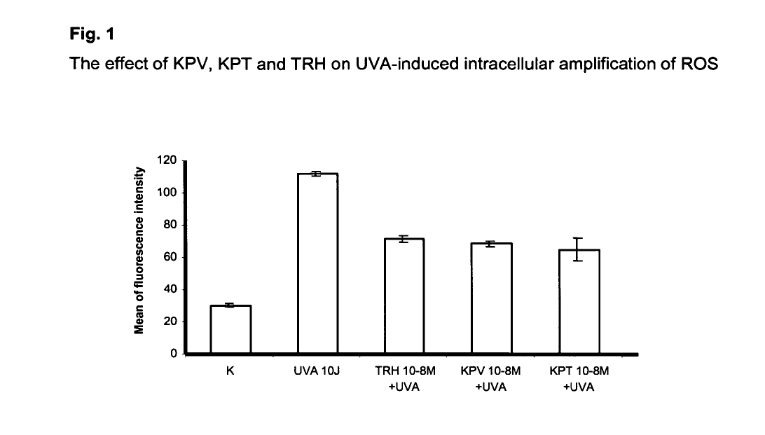

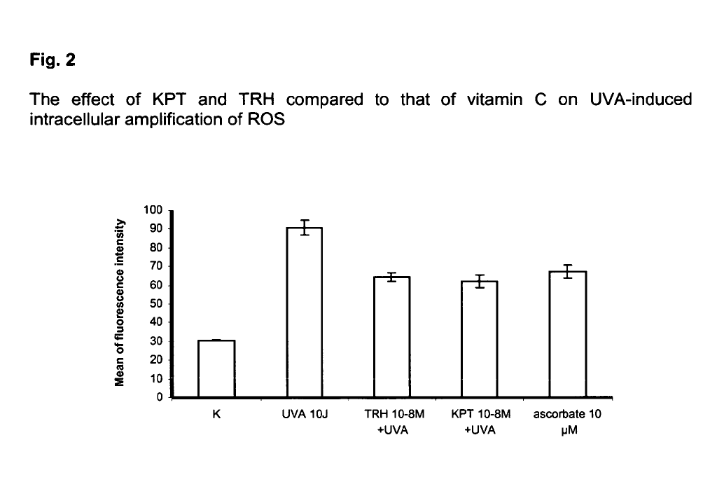

In a preferred embodiment it is envisaged that the compounds of the invention show an effect on UV-A induced intracellular amplification of ROS which is comparable to at least one of the compounds selected from the three test compounds of the invention which were evaluated in Example 1 and/or which is comparable to ascorbic acid, for example under conditions which equate with those exemplified in Example 1 (the results of this evaluation are depicted in FIGS. 1 and 2). “Comparable” means that the compounds of the invention suppress UV-A induced intracellular amplification of ROS with a deviation of the suppressing activity in respect to at least one, two, or three compound(s) selected from the three invention's compounds of Example 1 (and/or ascorbic acid) of not more than about 40%, 30%, 20%, 15%, 10%, 5%, 2.5%, 2% or 1%, preferably under conditions which equate to or are identical with those set out in Example 1. The skilled person is able to justify which assay conditions/assays equate with the assay/conditions exemplified in the appended examples. The effect of the compounds of the invention on UV-A induced intracellular amplification of ROS is thus determinable by the methods disclosed herein.

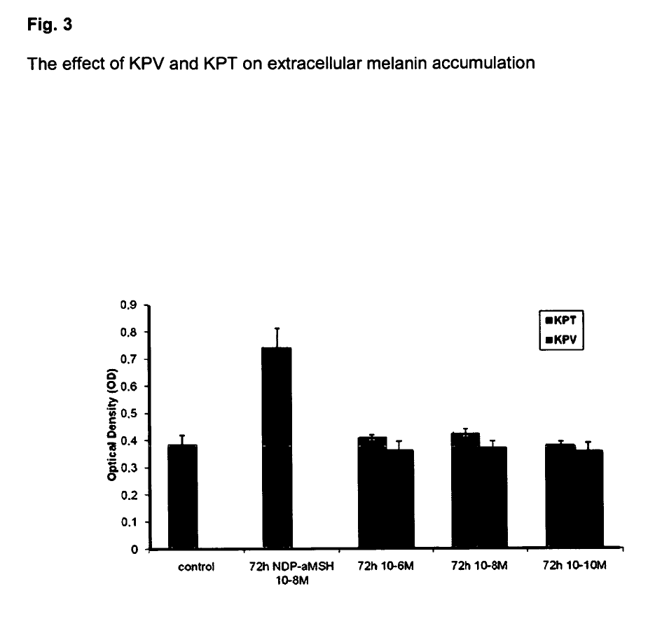

It is also preferred that the compounds of the invention have no or merely a negligible melanotropic effect. “Melanotropic effect” means that the compound induces the extracellular melanin accumulation in a cell assay in accordance with the method described in the appended example 2 and/or in accordance with the method described in Siegrist and Eberle (Anal. Biochem. 1986; 159; 191-197), title “In situ melanin assay for MSH using mouse B16 melanoma cells in culture”. “No or negligible” means that the mean melanin accumulation which is induced by the compounds of the invention exceeds the mean melanin accumulation which is induced by at least one of the two compounds as exemplified in Example 2 by not more than about 40%, 30%, 20%, 15%, 10%, 5%, 2.5%, 2% or 1%, preferably under conditions which equate to or are identical with those set out in Example 2. The skilled person is able to justify which assay conditions/assays equate with the assay/conditions exemplified in the appended examples. The melanin accumulation evaluated in Example 2 is depicted in FIG. 3. The effect of the compounds of the invention on extracellular melanin accumulation is thus determinable by the methods disclosed herein.

In a more preferred embodiment it is envisaged that the compounds of the invention have no or merely a negligible melanotropic effect and, at the same time, show an effect on UV-A induced intracellular amplification of ROS which is comparable to at least one of the compounds selected from the three test compounds of the invention which were evaluated in Example 1 and/or which is comparable to ascorbic acid, for example under conditions which equate with those exemplified in Example 1.

“Compounds of the invention” includes all variations of the tripeptides/compounds described herein (i.e. chemically modified, protected etc.).

The term “oxidative stress” as used herein particular relates to the effect of production of reactive oxygen species, for example to the intracellular increase of ROS. Reactive oxygen species (ROS) are generated in various tissues or cells (intracellular), such as fibroblasts, keratinocytes, melanocytes, cells of the hair follicle and epithelial layers of other non-cutaneous organs. ROS include oxygen ions, free radicals and peroxides both inorganic and organic. They are generally very small molecules and are highly reactive due to the presence of unpaired valence shell electrons. ROSs form as a natural byproduct of the normal metabolism of oxygen and have important roles in cell signaling. However, during times of environmental stress ROS levels can increase dramatically, which can result in significant damage to cell structures. This cumulates into a situation known as oxidative stress. Various types of radiation, like UV radiation, including UVA and UVB, or ionizing radiation, may induce oxidative stress. The present invention aims to reduce the intracellular ROS, and thereby to reduce the oxidative stress. Methods to determine intracellular ROS-production are known to the skilled person and to the more exemplified in the appended examples.

Thus, in one embodiment of the invention, the above compounds are used to reduce oxidative stress, whereby oxidative stress is induced by ultraviolet radiation, in particular UVA but also UVB. “Ultraviolet (UV) light” as used herein refers to electromagnetic radiation with a wavelength shorter than that of visible light, but longer than soft X-rays, including UVA and UVB. UVA, the long wave portion of UV light, also called black light, ranges from 400 nm-320 nm and UVB or medium wave UV light ranges 320 nm-280 nm. In humans, prolonged exposure to solar UV radiation may result in acute and chronic health effects on the skin, hair, eye, and immune system.

In a further embodiment of the invention, the disease or damage to be treated by the above compounds according to the invention is caused by ionizing radiation.

The term “ionizing radiation” as used herein means energetic particles or waves that have the potential to ionize an atom or molecule through atomic interactions. These ionizations, if enough occur, can be destructive to biological organisms, and can cause DNA damage in individual cells. Extensive doses of ionizing radiation have been shown to have a mutating effect to future generations of the individual receiving the dose. Examples of ionizing radiation are energetic beta particles, neutrons, alpha particles and energetic photons (UV and above).

The biological effects of ionizing radiation on living cells may result in a variety of outcomes including, for instance, that cells experience DNA damage and are unable to repair the damage. These cells may go through the process of programmed cell death, or apoptosis, thus eliminating the potential genetic damage from the larger tissue. Cells may experience a nonlethal DNA mutation that is passed on to subsequent cell divisions. This mutation may contribute to the formation of a cancer. For example, damage caused by ionizing radiation may be damages of the skin, mucosa, eye or the gonads, like necrosis or erythema.

According to the present invention, the above compounds are to be used as an active compound for use in the treatment of a disease or damage caused by oxidative stress and/or for the manufacture of a pharmaceutical composition for the treatment of a disease or damage caused by oxidative stress. The present invention is also directed to the use of the compounds as defined above for the manufacture of a pharmaceutical composition for the treatment of a disease or damage caused by oxidative stress. The treatment may be a prophylactic or therapeutic treatment.

The disease or damage to be treated with the pharmaceutical composition that contains the active compound characterized above is caused by oxidative stress, e.g by UV-induced oxidative stress, including UV-A and/or UV-B-induced oxidative stress. Oxidative stress may easily be determined by tests known to the person skilled in the art, such as the test described in Example 1. Examples for a disease or damage caused by oxidative stress are damages or diseases of the skin mucosa, eye or the gonads, like vitiligo, scleroderma, necrosis, or erythema; furthermore, a disease or damage of the hair, like premature hair loss or premature formation of grey hair, alopecia in general but also radiation- and chemotherapy-induced hair loss. The term “alopecia” circumscribes a plethora of attacks on the hair follicle having the consequence, whatever the reason, of the partial or general definitive loss of hair. Exemplary thereof are androgenetic alopecia, alopecia greata (pelade) or alopecia totalis, or alternatively alopecia universalis. “Hair loss”, when used in the context of the present invention, includes all forms of hair loss and specifically includes at least premature hair loss, radiation- and chemotherapy-induced hair loss and alopecia as defined herein before.

It is preferred that the herein defined ^{N}Lys-Pro-Val^{C} (including all its variations described herein, i.e. its protected form; chemically modified form etc) is not used as the sole active ingredient for the treatment of (for treating) hair loss.

In a preferred embodiment, the present invention, therefore, relates to a compound selected from the group consisting of
(a) ^{N}Lys-Pro-Thr^{C}; and
(b) ^{N}pGlu-His-Pro^{C}
   or to a combination of at least two compounds selected from the group consisting of
(c) ^{N}Lys-Pro-Val^{C};
(d) ^{N}Lys-Pro-Thr^{C}; and
(e) ^{N}pGlu-His-Pro^{C}
   for use in the treatment (for treating) hair loss. The compounds are defined herein elsewhere.

For the purpose of the invention the active compound as defined above also includes the pharmaceutically or cosmetically acceptable salt(s) thereof. The phrase “pharmaceutically or cosmetically acceptable salt(s)”, as used herein, means those salts of compounds of the invention that are safe and effective for the desired administration form. Pharmaceutically or cosmetically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The term “active compound” as used herein refers to the compounds according to the invention as defined herein.

The pharmaceutical compositions of the invention can be formulated in a manner known per se to the skilled person as described, for example, in Remington's Pharmaceutical Sciences, 15^{th} Ed., Mack Publishing Co., New Jersey (1991) and Bauer at al, Pharmazeutische Technologie, 5^{th} Ed., Govi-Verlag Frankfurt (1997).

Pharmaceutical compositions of the invention comprise a therapeutically effective amount of the compound of the present invention or a pharmaceutically acceptable salt thereof and can be formulated in various forms, e.g. in solid, liquid, powder, aqueous, lyophilized form. The pharmaceutical composition may be administered with a pharmaceutically acceptable carrier to a patient, as described herein. In a specific embodiment, the term “pharmaceutically acceptable” means approved by a regulatory agency or other generally recognized pharmacopoeia for use in animals, and more particularly in humans. Accordingly, the pharmaceutical composition may further comprise a pharmaceutically acceptable carrier or excipient.

Pharmaceutically acceptable carriers, which may be used in formulating the composition according the invention, comprise those described below for the cosmetic composition.

Other suitable pharmaceutically acceptable carriers and excipients are inter alia described in Remington's Pharmaceutical Sciences, 15^{th} Ed., Mack Publishing Co., New Jersey (1991) and Bauer at al, Pharmazeutische Technologie, 5^{th} Ed., Govi-Verlag Frankfurt (1997).

The administration of the pharmaceutical composition can be done in a variety of ways, including, but not limited to, topically, transdermally, subcutaneously, intravenously, intraperitoneally, intramuscularly or intraocularly. Preferably the pharmaceutical composition is to be administered topically.

The dose of the active compound is normally between concentrations of 1 nM and 1 mM, preferably between 1 μM and 100 μM.

In case of ordinary topical administration, the dose of the active compound may be in the range of 1 ng to 1 μg per cm²; skin per day or in several portions daily. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art. A typical dose can be, for example, in the range of 0.0001 to 100 μg kg body weight; however, doses below or above this exemplary range are envisioned, especially considering the aforementioned factors.

The pharmaceutical composition according to the invention may be in solid, liquid or gaseous form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for topical or oral administration.

A particular preferred form of administration of the pharmaceutical composition is topically, for instance in form of an ointment or cream. Such an ointment or cream may additionally comprise conventional ingredients, like carriers or excipients as described above.

The invention is further directed to the cosmetic use of the compounds according to the invention for the preparation of a composition for skin aging.

The term “cosmetic use” comprises the use of the active compound according to the invention in cosmetic compositions; such as care products for the skin. The cosmetic compositions include for example skin cosmetic preparations, such as W/O or O/W skin and body creams, day and night creams, light protection compositions, aftersun products, skin aging products, hand care products, face creams, multiple emulsions, gelees, microemulsions, liposome preparations, niosome preparations, antiwrinkle creams, face oils, lipogels, sportgels, moisturizing creams, bleaching creams, vitamin creams, skin lotions, care lotions, ampoules, aftershave lotions, preshaves, humectant lotions, tanning lotions, cellulite creams, depigmentation compositions, massage preparations, body powders, face tonics, deodorants, antiperspirants, nose strips, antiacne compositions, repellents and others.

The term “skin aging” as used in the context of the invention, includes the so-called “intrinsic” and “extrinsic” aging of the skin. The biological mechanism of said aging of the skin is characterized by an alteration of the dermis with appearance of folds and wrinkles, sagging and relaxing of the cutaneous tissue.

The main clinical signs of skin aging are the following:
(a) Appearance of deep wrinkles, increasing with age. A disorganization of the “grain” of the skin is noted, that is to say the micro-relief is less regular and is anisotropic in nature.
(b) The skin color is generally modified, appearing paler and yellower, which appears to be due chiefly to a disorganization of the microcirculation (less haemoglobin in the papillary layer of the dermis). Numerous colored spots appear at the surface, which is due to impaired melanogensis. On some areas, diffuse irritation and sometimes telangiectasia are present.
(c) Another clinical sign of aging is the dry and rough appearance of the skin, which is due chiefly to greater desquamation, these squamae contributing also to the somewhat grey appearance of the color by diffracting light rays.
(d) Finally, a loss is noted in firmness and tonus of the skin, which, as in the case of wrinkles, is explained at least partially by a dermal and epidermal atrophy as well as a flattening of the dermoepidermal formation.

Thus, as used herein “skin aging” means at least one clinical sign selected from the clinical signs explained above, i.e. selected from (a) appearance of deep wrinkles, (b) modification of color of the skin, (c) dryness and roughness of the skin and/or (d) a loss is noted in firmness and tonus of the skin. It is preferred that the above indicated clinical signs occur without the significant presence of inflammatory signs or other (inflammatory) disease pattern of the skin, more preferably in the absence, and even more preferably in the complete absence of any inflammatory signs or other disease pattern. “At least one” as used herein includes two or three or all four of the above indicated clinical signs, for example (a) and (b), or (a) and (c), or (c) and (b), or (a) and (b) and (c), or (a) and (b) and (d), or (b) and (c) and (d) etc.

In a further embodiment of the invention, skin aging is UV-induced photoaging. The term “photoaging” as used herein refers to the premature aging of the skin caused by UV-radiation. In particular, overexposure to the sun causes photoaging. Excessive UV radiation of the skin develops inter alia a leathery texture, wrinkles, skin folds, sagging skin, and warty growths called keratoses, freckling, and a yellow discolouration due to abnormal elastic tissue.

Accordingly, a further embodiment the invention is directed to a cosmetic composition comprising a compound of the invention as the active compound and a cosmetically acceptable carrier or excipient.

The cosmetic composition may be delivered in various ways, such as orally or topically. The cosmetic composition is preferably delivered topically. Topical administration of the cosmetic composition of the present invention is useful when the desired treatment involves areas or organs readily accessible by topical administration. For application topically to the skin, the cosmetic composition may be formulated with a suitable lotion, cream, gel, paste, ointment, or transdermal patches. The cosmetic can, depending on the field of use, also be in the form of a spray (pump spray or aerosol), foam, gel spray, mousse, suspensions or powders.

The cosmetic composition may be formulated with a suitable lotion or cream comprising the active components suspended or dissolved in a carrier. Such carriers include, but are not limited to, one or more of mineral oil such as paraffin, vegetable oils such as castor oil, castor seed oil and hydrogenated castor oil, sorbitan monostearate, polysorbate, fatty acid esters such as cetyl ester, wax, fatty acid alcohols such as cetyl alcohol, stearyl alcohol, 2-octyldodecanol, benzyl alcohol, alcohols, triglycerides and water.

Alternatively, the cosmetic composition may also be formulated with a suitable gel comprising the active components suspended or dissolved in a carrier. Such carriers include, but are not limited to, one or more of water, glycerol, propylene glycol, liquid paraffin, polyethylene, fatty oils, cellulose derivatives, bentonite and colloidal silicon dioxide.

Suitable propellants for aerosols according to the invention are the customary propellants, for example propane, butane, pentane and others.

A suitable paste comprises the active compound suspended in a carrier. Such carriers include, but are not limited to, petroleum, soft white paraffin, yellow petroleum jelly and glycerol.

The cosmetic composition may further comprise additional components, as are customarily used in such preparations, e.g. moisturizing substances, olfactory agents, emulsifiers, preservatives, perfumes, antifoams, dyes, pigments, thickeners, surface-active substances, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents, silicone derivatives, UV-filtering substances, or substances which absorb UV radiation in the UV-B and/or UV-A region.

The cosmetic composition according to the invention may preferably comprise moisturizing substances or emollients. Moisturizing substances or emollients may be used in amounts, which are effective to prevent or relieve dryness. Useful moisturizing substances or emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; ether esters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical moisturizing substances or emollients include mineral oil, especially mineral oils having a viscosity in the range of 50 to 500 SUS, lanolin oil, mink oil, coconut oil, cocoa butter, olive oil, almond oil, macadamia nut oil, aloa extract, jojoba oil, safflower oil, corn oil, liquid lanolin, cottonseed oil, peanut oil, purcellin oil, perhydrosqualene (squalene), caster oil, polybutene, odorless mineral spirits, sweet almond oil, avocado oil, calophyllum oil, ricin oil, vitamin E acetate, olive oil, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricinoleates of alcohols and polyalcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

Moreover, the cosmetic composition according to the invention may preferably comprise emulsifiers. Emulsifiers (i.e., emulsifying agents) are preferably used in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty acid monoesters containing sulfuric esters, e.g., glyceryl monostearate containing sodium lauryl surfate; (ii) cationics chloride such as N (stearoyl colamino formylmethyl)pyridium; N-soya-N-ethyl morpholinium ethosulfate; alkyl dimethyl benzyl ammonium chloride; diisobutylphenoxyethoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and ethoxylated cholesterol. The selection of emulsifiers is exemplarily described in Schrader, Grundlagen and Rezepturen der Kosmetika, Hüthig Buch Verlag, Heidelberg, 2^{nd} edition, 1989, 3^{rd} part.

The cosmetic composition of the present invention may preferably comprise a preservative. Preservatives used in compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

A cosmetic composition according to the invention may also comprise an olfactory agent or perfume. Olfactory agents, perfumes (fragrance components) and colorants (coloring agents) well known to those skilled in the art may be used in effective amounts to impart the desired fragrance and color to the compositions of the invention

The cosmetic composition according to the invention may also include a surfactant. Suitable surfactants may include, for example, those surfactants generally grouped as cleansing agents, emulsifying agents, foam boosters, hydrotropes, solubilizing agents, suspending agents and non-surfactants (facilitates the dispersion of solids in liquids).

The surfactants are usually classified as amphoteric, anionic, cationic and non-ionic surfactants. Amphoteric surfactants include acylamino acids and derivatives and N-alkylamino acids. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic acids; sulfonic acids and salts, such as, acyl isothionates, alkylaryl sulfonates, alkyl sulfonates, and sulfosuccinates; sulfuric acid esters, such as, alkyl ether sulfates and alkyl sulfates. Cationic surfactants include: alkylamines, alkyl imidazolines, ethoxylated amines, and quaternaries (such as, alkylbenzyldimethylammonium salts, alkyl betaines, heterocyclic ammonium salts, and tetra alkylammonium salts). Nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

Furthermore, a cosmetic composition according to the invention may also comprise a film former. Suitable film formers which are used in accordance with the invention keep the composition smooth and even and include, without limitation: acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like. Generally, film formers can be used in amounts of about 0.1% to about 10% by weight of the total composition with about 1% to about 8% being preferred and about 0.1 DEG/O to about 5% being most preferred.

Humectants can also be used in effective amounts, including: fructose; glucose; glutamic acid; glycerin; honey; maltitol; methyl gluceth-10; methyl gluceth-20; propylene glycol; sodium lactate; sucrose; and the like.

The invention is further directed to a method for the preparation of a cosmetic composition of the invention comprising the step of providing at least one of the compounds ^{N}Lys-Pro-Val^{C}, ^{N}Lys-Pro-Thr^{C} and ^{N}pGlu-His-Pro^{C} as defined herein and combining them with a cosmetically acceptable carrier or excipient. In the context of the present invention the term “combining” includes “mixing”.

Compositions according to the invention may be prepared according to methods well known to the person of ordinary skills in the art (see e.g. Bauer et al., Pharmazeutische Technologie, 5. edt. Govi-Verlag Frankfurt, 1997; Rudolf Voigt, Pharmazeutische Technologie, 9. edt., Deutscher Apotheker Verlag Stuttgart, 2000).

A cosmetic composition according to the invention comprises, for example O/W and W/O creams, O/W and W/O emulsions, gels, multiple emulsions (W/O/W and O/W/O), cosmetic dispersions (hydrodispersions and lipodispersions), sticks, formulations comprising a tenside or simple solutions (oily or aqueous).

An O/W formulation for the skin may be formulated by mixing, for example, the following ingredients in accordance with the International Nomenclature of Cosmetic Ingredients, INCI:
A ceteareth-6, stearyl alcohol, ceteareth-25, diethylamino hydroxybenzoyl hexyl benzoate, PEG-14 dimethicone, cetearyl alcohol, ethylhexyl methoxycinnamate, dibutyl adipate;
B glycerol, panthenol, preservative, aqua dem;
C caprylic/capric triglyceride, sodium acrylates copolymer;
D sodium ascorbyl phosphate, tocopheryl acetate, bisabolol, caprylic/capric triglyceride, sodium ascorbate, tocopherol, retinol; active compound; and
E sodium hydroxide

Phases A and B are separately heated. Phase B is subsequently stirred into phase A and homogenized. Phase C is stirred into a combination of phases A and B and homogenized. The mixture is under agitation cooled down; then phase D is added and the pH is adjusted with phase E. The solution is subsequently homogenized and cooled down to room temperature.

The exact amount of the particular ingredients and conditions may vary dependent on the particular application and administration form. The person skilled in the art is able to easily determine the exact amount and condition given the specification and references therein.

The Figures show:

FIG. 1: The effect of KPV, KPT and TRH on UVA-induced intracellular amplification of ROS

FIG. 2: The effect of KPT and TRH on UVA-induced intracellular amplification of ROS compared to that of vitamin C

FIG. 3: The effect of KPV and KPT on extracellular melanin accumulation

A better understanding of the present invention and of its advantages will be had from the following example, offered for illustrative purposes only, and are not intended to limit the scope of the present invention in any way.

### EXAMPLE 1

### The Effect of KPV, KPT and TRH on UVA-Induced Intracellular Amplification of ROS

### Methods

The modulating effect of the tripeptides Lys-Pro-Val (KPV), Lys-D-Pro-Thr (KPT) and Thyrotropin Releasing Hormones (TRH, Protirelin, Thyoliberin, pGlu-His-Pro) was examined on human dermal fibroblasts that were seeded into 3.5 cm² tissue culture dishes in a density of 150,000 per dish in RPMI 16MI medium. Two days after seeding the cells, the medium supplemented with 10% foetal calf serum, 1% L-glutamine and 1% penicillin/streptomycin was exchanged with serum-free RPMI medium and pre-incubated for 24 hours with KPV, KPT or TRH in a concentration of 10⁻⁸ M or with ascorbic acid in a concentration of 10 μM. Subsequently, the cells were exposed to a physiological dose of UVA radiation (10 J/cm²). Immediately after radiation, the cells were incubated with 5 μM dihydrorhodamine 123 plus 5 mM glucose in phosphate buffer (PBS) for 30 minutes. Subsequently, the cells were trypsinized, resuspended in PBS with 5 mM glucose and analyzed by means of flow cytometry (FACS, FACSCalibur with 488 nm argon laser). The above dihydrorhodamine probe is oxidized in the presence of intracellular ROS into green fluorescent dihydrorhodamine 123 and, thus, can be measured fluorometrically. A total of 10×10³ cells per probe were analyzed and evaluated using CELL-QUEST software. The median of the FL-1 channel was used as parameter for the intracellular ROS amount, since it represents the maximum number of cells with the highest fluorescence. Duplicate and triplicate analyses were carried out and all assays were reproduced three times in independent experiments.

### Result

All three tripeptides tested, KPV, KPT and TRH, suppressed UVA-induced intracellular amplification of ROS (“oxidative stress”) in a significant way (FIG. 1, p<0.001). The effect of KPT and TRH (when used in a concentration of 10-8M) on UVA-induced intracellular amplification of ROS was comparable with that of vitamin C (ascorbate) which was used in a significantly higher concentration, namely 10-2M. (FIG. 2)

### EXAMPLE 2

### The Effect of KPV and KPT on Extracellular Melanin Accumulation

To exclude a potential melanotropic effect of KPT and KPV, 2500 B16.F1 melanoma cells were seeded out on 96-well tissue culture plates in quintuplicate at a density of 2500 cells/well in regular culture medium. On the next day routine medium was changed to medium containing the above peptides at 10⁻⁶, 10⁻⁸ and 10⁻¹⁰ M vs. medium containing the superpotent MSH analogue NDP-α-MSH (10⁻⁸ M). The latter served as positive control while cells without any other stimulus served as negative control. Cells were then cultured for 72 hrs followed by photometric measurement of the optical density (wavelength 405 nm) of each well. This procedure measures the amount of extracellular melanin produced by the cells according to the well-established and described methodology by Siegrist & Eberle, Anal. Biochem. 1986; 159: 191-197. In contrast to NDP-α-MSH (p<0.001 vs. control) both KPV and KPT did not have any melanotropic effect.

1-15. (canceled)

16. A cosmetic composition comprising a compound selected from the group consisting of
(a) ^{N}Lys-Pro-Val^{C};
(b) ^{N}Lys-Pro-Thr^{C}; and
(c) ^{N}pGlu-His-Pro^{C}
and a cosmetically acceptable carrier or excipient.

17. The cosmetic composition of claim 16, in which is in form of a lotion, gel, spray or cream.

18. The cosmetic composition of claim 16, characterized in that the carrier or excipient is selected from the group consisting of moisturizing substances, olfactory agents, emulsifiers and preservatives.

19. A method for the preparation of a cosmetic composition of claim 16 comprising the step of providing the compound selected from the group consisting of
(a) ^{N}Lys-Pro-Val^{C};
(b) ^{N}Lys-Pro-Thr^{C}; and
(c) ^{N}pGlu-His-Pro^{C}
and combining them with a cosmetically acceptable carrier or excipient.

20-23. (canceled)

24. A method of treating a patient by administration of a composition comprising at least one compound selected from the group consisting of
(a) ^{N}Lys-Pro-Val^{C};
(b) ^{N}Lys-Pro-Thr^{C}; and
(c) ^{N}pGlu-His-Pro^{C}
in an amount effective for the reduction of oxidative stress.

25. The method of claim 24, wherein the amino acids of compound (a), (b) or (c) have either (L) or (D) configuration.

26. The method of claim 24, wherein the compound is selected from the group consisting of
(a) (L)Lys-(L)Pro-(L)Val;
(b) (L)Lys-(D)Pro-(L)Thr; and
(c) (L)pGlu-(L)His-(L)Pro.

27. The method of claim 24, wherein the compound is chemically modified by alkylation, arylation, etherification and esterification.

28. The method of claim 24, wherein the compound is acetylated at the N-terminus and/or amidated or esterified at the C-terminus.

29. The method of claim 24, wherein the oxidative stress is UVA-induced.

30. The method of claim 24, wherein the patient is treated for a disease or damage caused by oxidative stress.

31. The method of claim 30, wherein the disease or damage is caused by UVA-induced oxidative stress.

32. The method of claim 30, wherein the disease or damage is vitiligo, scleroderma, hair loss or premature formation of grey hair.

33. The method of claim 30, wherein the disease or damage is caused by ionizing radiation.

34. The method of claim 24, wherein the composition is to be administered topically.

35. The method of claim 24, wherein the composition is in form of an ointment or cream.

36. The method of claim 24, wherein the active ingredient is to be administered in an amount of about 1 nM to 1 mM.

37. The method of claim 24, wherein the composition is a cosmetic composition for skin aging.

38. The method of claim 37, wherein the skin aging is UV-induced photoaging.
